(19) United States
(12) Patent Application Publication
(10) Pub. No.: US 2025/0123292 A1
(43) Pub. Date: Apr. 17, 2025

(54) LG3 Assay

(71) Applicant: Nordic Bioscience A/S, Herlev (DK)

(72) Inventors: Clara Fia Gøricke Laursen, København N (DK); Nadja Sparding, Kongens Lyngby (DK); Alexandra Louise Møller, Humlebæk (DK); Federica Genovese, Valby (DK); Lasse Langholm, Ølstykke (DK); Morten Asser Karsdal, Kobenhavn Ø (DK)

(73) Assignee: Nordic Bioscience A/S, Herlev (DK)

(21) Appl. No.: 18/485,767

(22) Filed: Oct. 12, 2023

(51) Int. Cl. G01N 33/68

(52) U.S. Cl. CPC G01N 33/6893; G01N 2800/347

(57) ABSTRACT

Disclosed herein are methods of immunoassay for detecting, in a patient sample, the third laminin-like globular domain (LG3) fragment cleaved and released from endorepellin/perlecan by bone morphogenetic protein-1 (BMP-1). Also disclosed are monoclonal antibodies and immunoassay kits for use in these methods of immunoassay, as well as the use of these methods of immunoassay for detecting and/or monitoring fibrotic kidney diseases, such as Lupus Nephritis (LN) or IgA nephropathy (IgAN), or particular levels of severity thereof.

### SEQUENCE LISTING

A sequence listing is electronically submitted in XML format in compliance with 37 C.F.R. § 1.831(a) and is incorporated by reference herein. The XML file is named D7983SEQ, was created on Oct. 10, 2023 and is 12 KB in size.

### BACKGROUND OF THE INVENTION

### Field of the Invention

The present invention relates to methods of immunoassay for detecting, in a patient sample, the third laminin-like globular domain (LG3) fragment of endorepellin/perlecan cleaved and released from endorepellin/perlecan by bone morphogenetic protein-1 (BMP-1). The present invention also relates to monoclonal antibodies and immunoassay kits for use in said methods of immunoassay, and to the use of said methods of immunoassay for detecting and/or monitoring a fibrotic kidney disease or level of severity thereof. In particular, the fibrotic kidney disease may be Lupus Nephritis (LN) or IgA nephropathy (IgAN).

### Description of the Related Art

Fibrotic kidney diseases constitute a major health problem worldwide due to the large number of affected individuals, the incomplete knowledge of the pathogenesis, and the heterogeneity in their etiology and clinical manifestations, as well as the absence of accurate biomarkers and effective disease-modifying therapeutic agents. Fibrosis is driven by dysregulated extracellular matrix (ECM) remodeling and is associated with a variety of chronic diseases. Accurate fibrosis assessment often requires invasive tissue biopsies that may only detect the injury when organ function has already been lost. Therefore, there is a need for non-invasive biomarkers to complement or replace kidney biopsies and aid in the evaluation of therapeutic compounds.

Together with collagens, proteoglycans constitute the most abundant proteins in the ECM, providing well-organized non-cellular structural networks supporting cells and tissues in a highly specialized and dynamic matter. Perlecan is a large heparan sulfate proteoglycan (HSPG) located in the basement membrane in the ECM of endothelial and epithelial cells in various tissues (1), regulating physiological and pathological processes including organ development and wound healing. Moreover, perlecan plays vital roles in structural integrity and maintaining tissue and cellular homeostasis, which is achieved by interacting with and signaling to a range of ligands such as integrins, growth factors, adhesion molecules and collagens inducing proliferation and angiogenesis (1). The structure of perlecan is shown in FIG. 1.

Proteolytic cleavage of the C-terminal end of perlecan releases the signaling molecule named endorepellin, which contains three laminin-like globular (LG) domains inter-connected by epidermal growth factor-like (EGF-like) repeats. In contrast to full-length perlecan, endorepellin has different anti-angiogenic and apoptotic properties, including inhibiting endothelial cell migration, collagen-induced endothelial tube morphogenesis and blood vessel growth (2). The third LG domain (LG3) of endorepellin has been shown to harbor most of the apoptotic and anti-angiogenic activity on endothelial cells (3). Gonzales et al. showed by MS analysis that cleavage of endorepellin/perlecan between Asn4196 and Asp4197 (amino acid numbering according to the full perlecan sequence) by bone morphogenetic protein-1 (BMP-1)/Tolloid-like family of metalloproteases, liberates the LG3 fragment identical to the fragments generated in vivo (4). In addition, the specificity of the cleavage site was confirmed by mutation of Asp4197 preventing the processing of endorepellin. Moreover, BMP-1 was demonstrated to cleave at the specific site with highest activity level compared to mTLL-1, MTLL-2 and mTLD (4).

The LG3 fragment was first identified and characterized in urine from end-stage renal failure confirmed by the molecular weight of 25 kDa corresponding to the calculated weight of the reported DNA sequence for the third LG domain of perlecan (5). The LG3 fragment has since shown to be associated with acute vascular rejection and adverse outcomes after kidney transplantation (6). In a previous study by Surin et al. 2013 (7), the LG3 fragment was evaluated as a potential biomarker for IgA Nephropathy severity, where LG3 fragment levels were analyzed by western-blotting and tandem mass spectrometry. An ELISA (using a commercial polyclonal anti-human endorepellin antibody (R&D systems)) was also used, in order to quantify the levels of the LG3 fragment in the urine of healthy controls and IgAN patients (7). The above study found that the levels of the LG3 fragment were significantly reduced in urine of patients with IgAN compared to healthy controls. However, a group of ten patients had increased LG3 fragment levels. These ten patients had a lower glomerular filtration rate and thus more severe progression. Increased levels of LG3 autoantibodies have been observed in LN, contributing to the characteristic microvascular damage (8). In addition, LG3 has been proposed as a potential serological biomarker for breast cancer, as significantly lower plasma levels of LG3 were observed in breast cancer patients compared to healthy donors, identified by proteomics (9).

To date it has not been evidently possible to distinguish the LG3 fragment from other C-terminal derived fragments from perlecan such as endorepellin or intact perlecan using enzyme linked immunosorbent assay (ELISA) (6).

Previously, another biomarker assay named uPERLA (Nordic Bioscience A/S) has been developed, targeting an N-terminal fragment 93‘SIEYSPQLED’102 (SEQ ID NO: 1) of perlecan cleaved by ADAM-TS4. However, this marker only works in urine and has shown to be losing activity from long term storage. Moreover, the assay is not very specific as it shows cross reactivity towards elongated peptide and has quite low sensitivity with a standard curve starting at 2000 ng/ml.

### SUMMARY OF THE INVENTION

The present inventors have now developed a robust and specific enzyme-linked immunosorbent assay (ELISA) targeting the LG3 fragment cleaved and released from endorepellin/perlecan by BMP-1, that may be used to detect the amount of LG3 fragment (also referred to herein as simply “LG3 levels” or “levels of LG3”) present in patient samples, and have demonstrated that significantly increased levels of LG3 are present in LN and IgAN patient samples (as compared to healthy controls), with higher levels being associated with more severe disease.

Accordingly, in a first aspect the present invention provides a method of immunoassay, the method comprising: contacting a patient sample with a monoclonal antibody that specifically binds to the N terminus amino acid sequence DAPGQYGAYF (SEQ ID NO: 2) (i.e. the 10 amino acid N-terminus sequence of the LG3 fragment formed by cleavage of endorepellin/perlecan by BMP-1 between Asn4196 and Asp4197 of the perlecan sequence, the N terminus amino acid sequence also being referred to here as the “LG3 target sequence” or simply the “target sequence”); and detecting and determining the amount of binding between said monoclonal antibody and peptides in the sample.

In a preferred embodiment, the method of immunoassay is a method of immunoassay for detecting and/or monitoring a fibrotic kidney disease or a particular level of severity thereof in a patient, the method further comprising correlating said amount of binding with values associated with normal healthy subjects and/or values associated with known disease severity and/or values obtained from said patient at a previous time point and/or with a predetermined cut-off value.

In a preferred embodiment, the fibrotic kidney disease is Lupus Nephritis (LN) or IgA nephropathy (IgAN), most preferably Lupus Nephritis (LN). The method may, in particular, be a method for detecting a particular level of severity of LN in a patient. For example, the method may be a method for detecting class IV+IV/V LN, or class IV+IV/V-global LN.

In a preferred embodiment, the patient sample is a human biofluid sample. Preferably the sample is urine or a blood-based sample such as blood (whole blood), plasma or serum. Most preferable the sample is a urine sample.

In a preferred embodiment, the monoclonal antibody does not specifically bind to a peptide having the N-terminus amino acid sequence NDAPGQYGAYF (SEQ ID NO: 3) (i.e. an elongated version of the target sequence extended at its N-terminus by the addition of an asparagine residue). Preferably, the ratio of the affinity of said antibody for said target sequence to the affinity of said antibody for the elongated version of the target sequence is at least 10 to 1, and more preferably is at least 20 to 1, at least 30 to 1, at least 40 to 1, at least 50 to 1, at least 75 to 1, or at least 100 to 1.

In a preferred embodiment, the monoclonal antibody not specifically bind to a peptide having the N-terminus amino acid sequence APGQYGAYF (SEQ ID NO: 4) (i.e. a truncated version of the target sequence truncated at its N-terminus by the removal of the initial aspartic acid residue). Preferably, the ratio of the affinity of said antibody for said target sequence to the affinity of said antibody for the truncated version of the target sequence is at least 10 to 1, and more preferably is at least 20 to 1, at least 30 to 1, at least 40 to 1, at least 50 to 1, at least 75 to 1, or at least 100 to 1.

In a preferred embodiment, the monoclonal antibody is raised against a synthetic peptide having the N-terminus amino acid sequence DAPGQYGAYF (SEQ ID NO: 2). For example, the monoclonal antibodies may be raised by: (a) immunizing a rodent (or other suitable mammal) with a synthetic peptide comprising the N-terminus sequence DAPGQYGAYF (SEQ ID NO: 2), which peptide may optionally be linked at its C-terminus to an immunogenic carrier protein (such as keyhole limpet hemocyanin (“KLH”)); (b) isolating and cloning a single antibody producing cell; and (c) assaying the resulting monoclonal antibodies to ensure that they have the desired specificity. An exemplary protocol of the development, production and characterization of suitable monoclonal antibodies is described in the Examples section, infra.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

In a second aspect, the present invention provides a method of a treating fibrotic kidney disease in a patient in need thereof, the method comprising: (a) carrying out a method of immunoassay for detecting a fibrotic kidney disease or a particular level of severity thereof in accordance with the first aspect of the present invention on a sample from a patient; and (b) administering to the patient a therapy for the treatment of the fibrotic kidney disease if it is determined in step (a) that the patient has said fibrotic kidney disease or said particular level of severity thereof.

In a third aspect, the present invention provides a monoclonal antibody that specifically binds to the N-terminus amino acid sequence DAPGQYGAYF (SEQ ID NO: 2).

In a fourth aspect, the present invention provides an immunoassay kit comprising a monoclonal antibody in accordance with the third aspect of the present invention, and at least one of: a streptavidin coated well plate; a biotinylated peptide DAPGQYGAYF-L-Biotin (SEQ ID NO: 5) wherein L is an optional linker; a secondary antibody for use in a sandwich immunoassay; calibrator protein comprising the N-terminus amino acid sequence DAPGQYGAYF (SEQ ID NO: 2); an antibody biotinylation kit; an antibody HRP labelling kit; or an antibody radiolabelling kit.

### BRIEF DESCRIPTION OF THE DRAWINGS

FIG. 1 illustrates the protein structure of perlecan. The five different domains of perlecan are: (i) the Sperm protein Entero-kinase and Agrin module (SEA) containing three heparan sulfate (HS) chains; (ii) four low density lipoprotein (LDL) receptor like repeats; (iii) laminin type B and EGF-like repeats; (iv) multiple immunoglobulin repeats; (v) three laminin-like globular domains (LG1-3) inter-connected by epidermal growth factor-like (EGF-like) repeats, that together form the signaling molecule named endorepellin. Cleavage by BMP-1 liberates the C-terminal LG3 domain from endorepellin.

FIG. 2 shows the specificity of the LG3 mAb and assay. Inhibition curves for the standard peptide, elongated peptide, truncated peptide, de-selection peptide, nonsense standard peptide and nonsense coater peptide are shown. Data is presented as the percentage of background fluorescence (buffer), as a function of peptide concentration (ng/ml) on a linear scale.

FIG. 3 is an in vitro cleavage experiment showing the measured levels of LG3 in samples originally containing endorepellin with BMP-1, BMP-1 only, endorepellin only, or BMP-1 with buffer. LG3 was detected only in the endorepellin with BMP-1 sample, demonstrating the specificity of the LG3 assay for the cleavage site of endorepellin with BMP-1 and recognition by the LG3 assay of the LG3 fragment and not the intact endorepellin.

FIGS. 4A-4D show levels of LG3 measured by the LG3 assay in serum (FIG. 4A-4B) or urine (FIG. 4C) from patients with lupus nephritis or IgA Nephropathy compared to health controls or levels of uPERLA in urine from patients with lupus nephritis (FIG. 4D). Levels of LG3 were significantly (p<0.001) increased in serum and urine (corrected for urinary creatinine) from patients with lupus nephritis (LN) or IgA Nephropathy (IgAN) compared to matched healthy controls. The same LN urine samples were evaluated with the former Nordic Bioscience perlecan marker uPERLA, where no significant difference was observed. Results are shown as Tukey's box and whiskers; non-parametric t-test (Mann Whitney test). *=p<0.05; **=p<0.01; ***=p<0.001; ****=p<0.001, NS: non-significant. Measurement range LLOQ-ULOQ.

FIGS. 5A-5D show measured levels of LG3 in urine and serum in LN patients divided into groups according to classification (FIGS. 5A-5B) and into diffuse segmental and global subgroups (FIGS. 5C-5D). Group 1 includes classes 11,111, and V (Urine: n=27, serum: n=24) and group 2 includes class IV and IV/V (urine: n=25, serum: n=24), being the most common and more severe class. Group 3 includes class VI(n=1) (FIGS. 5A-5B). Measured levels of LG3 in class IV or IV/V were further divided into diffuse segmental (S) (urine: n=10, serum: n=9) and IV-global(G) (urine and serum: n=11) LN subtypes (FIGS. 5C-5D). Type IV-G is known to be the class with the worst prognosis and the highest risk of ESRD. Results are shown as Tukey's box and whiskers; Mann-Whitney test and Kruskal-Wallis test followed by Dunn's multiple-comparison test. * p<0.05; ** p<0.01; **** p<0.0001; ns: non-significant.

FIGS. 6A-6D show levels of LG3 measured by the LG3 assay (FIGS. 6A-6B) and the endorepellin ELISA kit (RayBiotech) (FIGS. 6C-6D) in serum or urine from patients with lupus nephritis compared to health controls. Levels of LG3 were significantly (p<0.001) increased in serum (FIG. 6A) and urine (corrected for urinary creatinine) (FIG. 6B) from patients with lupus nephritis (LN) compared to matched healthy controls. The same serum (FIG. 6C) and urine (FIG. 6D) LN samples evaluated with the commercial endorepellin ELISA kit could differentiate between LN patients and healthy controls, but to a much lesser extent (p<0.05) than the LG3 assay. Results are shown as % of healthy, Mean SD; non-parametric t-test (Mann Whitney test). *=p<0.05; **=p<0.01; ***=p<0.001; ****=p<0.001. LLOQ for LG3 is 0.35 ng/ml for serum and 0.12 ng/ml for urine. LLOQ for the commercial endorepellin ELISA kit from RayBiotech is 0.08 ng/ml.

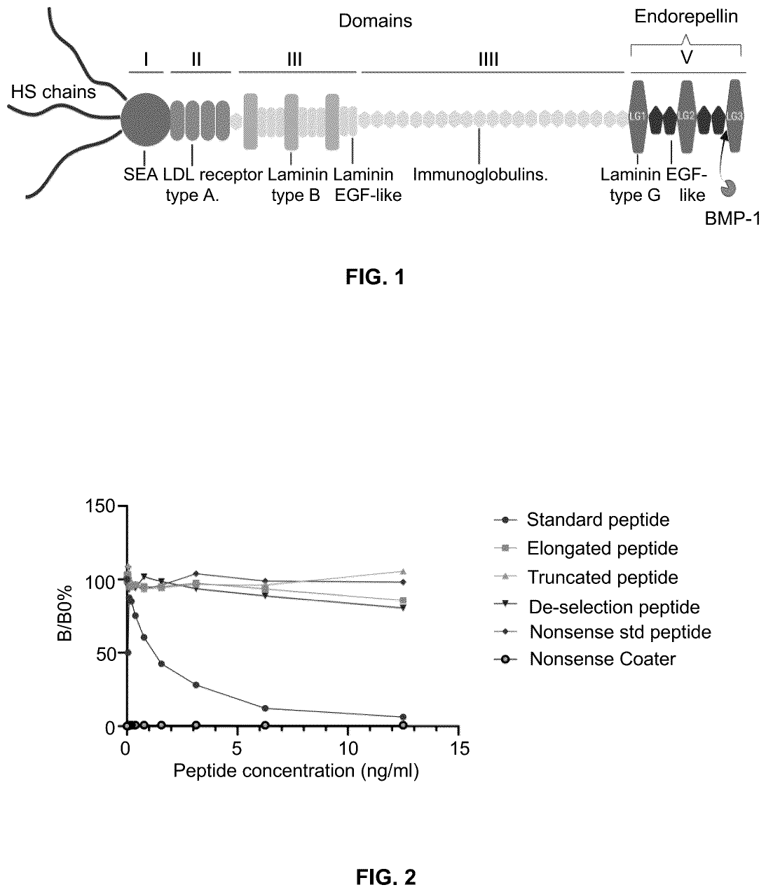

### DETAILED DESCRIPTION OF THE INVENTION

As used herein, unless expressly otherwise indicated in this specification, the word ‘or’ is used in the sense of an operator that returns a true value when either or both of the stated conditions is met, as opposed to the operator ‘exclusive or’ which requires that only one of the conditions is met.

As used herein, the term “comprising” is used to mean including or consisting of.

As used herein, the term “N-terminus” refers to an N-terminal peptide sequence at the extremity of a polypeptide, i.e. at the N-terminal end of the polypeptide, and is not to be construed as meaning in the general direction thereof. As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. Any suitable analytical method may be used for measuring the amount of binding. For example, an ELISA method may be used in which spectrophotometric analysis is used to measure the amount of binding both in the patient samples and when producing the calibration curve.

As used herein the term “predetermined cut-off value” means an amount of binding that is determined statistically to be indicative of a high likelihood of a disease (e.g. a fibrotic kidney disease) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or particular severity thereof.

As used herein, the term “values associated with normal healthy subjects” means standardised quantities of binding determined by the method described supra for samples from subjects considered to be healthy, i.e. without disease (i.e. without fibrotic kidney disease); and the term “values associated with known disease severity” means standardised quantities of binding determined by the method described supra for samples from patients known to have disease (i.e. a fibrotic kidney disease) of a known severity.

Provided in the present invention are methods, monoclonal antibodies and kits for the treatment of fibrotic kidney disease as summarized in the embodiments, preferred embodiments and aspects thereof as described herein. In the present invention, a monoclonal antibody comprising any constant region known in the art may be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGl, IgG2, IgG3, and IgG4 in the case of humans and IgGl, IgG2a, IgG2b, IgG2c and IgG3 in the case of mice. The monoclonal antibody may preferably be of the IgG isotype, including any one of the IgG subclasses.

The CDR of an antibody may be determined using methods known in the art such as that described by Kabat et al. Antibodies may be generated from B cell clones. The isotype of the antibody may be determined by ELISA specific for IgM, IgG or IgA isotype, or subclass. The amino acid sequence of the antibodies generated may be determined using standard techniques. For example, RNA may be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences may be used together with primers that bind to a sequence located in the constant region of the isotype which has been previously determined. The light chain may be amplified using primers which bind to the 3′ end of the Kappa or Lambda chain together with primers which anneal to the V kappa or V lambda leader sequence. The full length heavy and light chains may be generated and sequenced.

The therapy may be any therapy suitable for treating the fibrotic kidney disease in question. The therapy may for example comprise or consist of one or more surgeries, one or more medicaments, or combinations thereof. Medicaments may be formulated for enteral administration (e.g. oral, rectal or sublingual) or parenteral administration (e.g. via injection, inhalation, or application to the skin or a mucous membrane), and may for example be formulated as tablets, capsules, powders, mixtures, emulsions, gels, liquids, suspensions, creams, foams, lotions, ointments, or pills. Surgeries may be curative, preventative, palliative and/or restorative surgeries.

Where, for example, the fibrotic kidney disease is Lupus Nephritis (LN), suitable therapies may for example comprise one or more medicaments selected from: one or more corticosteroids; and one or more immunosuppressant drugs such as (but not limited to) mycophenolate mofetil (MMF), cyclophosphamide or azathioprine. Where, for example, the Lupus Nephritis is class IV+IV/V Lupus Nephritis, the medicaments may comprise in particular a combination of one or more corticosteroids and one or more immunosuppressant drugs.

Where, for example, the fibrotic kidney disease is IgA nephropathy (IgAN), suitable therapies may comprise, for example, one or more medicaments selected from one or more corticosteroids, one or more immunosuppressant drugs (such as for example cyclophosphamide), one or more angiotensin converting enzyme inhibitors (ACE inhibitors), one or more angiotensin receptor blockers (ARBs), and one or more sodium-glucose co-transporter-2 (SGLT2) inhibitors.

The antibody according to the third aspect of the invention is, in particular, suitable for use in carrying out the methods of immunoassay according to the first aspect of the invention. Preferred embodiments and features of the antibody according to the third aspect are therefore apparent from the above discussion of the preferred embodiments of the methods according to the first aspect.

The immunoassay kit according to the fourth aspect of the invention is, in particular, suitable for use in carrying out the method of immunoassay according to the first aspect of the invention. Further preferred embodiments and features of the immunoassay kit according to the fourth aspect are therefore apparent from the above discussion of the preferred embodiments of the methods according to the first aspect.

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Materials and Methods

The LG3 ELISA described herein was developed based on the principles of Nordic Bioscience Protein Fingerprint Technology™. This technology utilizes monoclonal antibodies developed against neo-epitopes generated by protease cleavage of ECM proteins. Importantly, these protein modifications are regulated by physiological or pathological mechanisms making them more likely to reflect disease activity (10). The technology is based on the recognition of neo-epitopes by monoclonal antibodies developed to target specific fragments of ECM proteins generated during tissue formation, degradation, or other post translational modifications in different pathogenesis. Targeting these fragments resembling changes in this equilibrium as opposed to measuring total protein levels may allow more precise evaluation of the mechanisms behind a pathology to monitor disease prediction, progression or evaluate new clinical approaches (11).

### Reagents

All reagents used for the experiments were standard high-quality chemicals from companies such as Sigma Aldrich (St. Louis, MO, USA), Merck (Whitehouse Station, NJ, USA), and Roche (Basel, Switzerland). The synthetic peptides used for monoclonal antibody production and assay development were purchased from Genscript (Piscataway, NJ, USA).

### Monoclonal Antibody Development and Production

The specific 10 amino acid (aa) target sequence 4197‘-DAPGQYGAYF-’4206 (SEQ ID NO: 2) (corresponding to the 10 aa N-terminus sequence of the LG3 fragment formed by cleavage of endorepellin/perlecan by BMP-1 between Asn4196 and Asp4197 of the perlecan sequence) was chosen for the development of monoclonal Abs (mAbs) based on the study by Gonzales et al. from 2005 (4), showing that BMP-1 cleaves at the specific site with high activity leaving the N-terminal end of LG3 accessible for Ab recognition and binding.

Antigen in the form of a synthetic peptide (DAPGQYGAYF (SEQ ID NO: 2)) corresponding to the target sequence was ordered from GenScript, and an immunogenic peptide was generated by covalently cross-linking the target peptide to Keyhole Limpet Hemocyanin (“KLH”) carrier protein using sulfosuccinimidyl 4-(N-maleimidomethyl) cyclohexane-1-carboxylate, SMCC (Thermo Scientific, Waltham, MA, USA, cat.no. 22322), with glycine and cysteine residues being added at the C-terminal end of the target peptide as a linker to ensure correct linking of the carrier protein to the target peptide. 100 μg of said immunogenic peptide (DAPGQYGAYF-GGC-“KLH” (SEQ ID NO: 6)) in 200 μl of Sigma adjuvant system (Sigma) was injected into six-seven weeks old Balb/C female mice, repeated every fourth week until stable levels of antibodies could be detected in the blood. The mice with the highest and best reactivity serum titer were selected for intravenous boosting. Three days prior to isolation of the spleen the mice were boosted intravenously with 100 μg immunogenic peptide in 200 μL 0.9% NaCl solution. Antibody-producing spleen cells were then isolated and fused with cultured myeloma cells, creating immortalized hybridomas, as described by Köhler et al. in 1975 (12). Hybridomas producing the specific antibodies were then screened after 14 days of culture, and positive cells were sub-cloned by limiting dilution for amplification (13). The supernatant (500 ml) from the antibody-producing hybridomas (monoclonal cells) was then collected for purification. Prior to purification, the presence of antibodies in the supernatant was tested in a competitive ELISA. The purification was carried out by using affinity chromatography by HiTrap protein G columns (Cytvia) on the ÄKTA Start system. HiTrap columns were prepacked with Q Sepharose resin with an affinity for IgG's, facilitating the separation of IgG from other proteins. The purified monoclonal antibody (mAb) was labelled with horse-radish peroxidase (HRP) using a peroxidase labelling kit (Roche, cat. no. 11829696001) according to the manufacturer's instructions. To evaluate the specificity of the selected mAb, the binding of the mAb to standard (DAPGQYGAYFHDDGFLAFPGHVFSRSLPEV (SEQ ID NO: 7)), elongated (NDAPGQYGAYF (SEQ ID NO: 3)), truncated (APGQYGAYF (SEQ ID NO: 4)) and other unrelated non-sense-peptides (see the “technical evaluation section below) was tested.

### LG3 ELISA Protocol

To determine the optimal settings for the ELISA, different parameters were tested: the concentration of coater/antibody, buffer, incubation time, and incubation temperature. The final established LG3 assay protocol (also referred to herein as the “LG3 assay”) employing the mAb described above and targeting the N-terminus amino acid sequence DAPGQYGAYF was as follows.

A 96-well streptavidin-coated white plate (Greiner) was coated with 100 μL/well of 1.5 ng/mL biotinylated (DAPGQYGAYF-K-Biotin (SEQ ID NO: 8)) peptide dissolved in coating buffer (25 mM TBS-BTB, 2 g/L NaCl, pH 8.0) and incubated for 30 min at 20° C. while shaken at 300 rpm. Following a washing cycle in washing buffer (25 mM Tris, 50 mM NaCl, pH 7.2), 20 μL of 12.5 ng/ml of the 30AA standard peptide (DAPGQYGAYFHDDGFLAFPGHVFSRSLPEV (SEQ ID NO: 7)) in a two-fold serial dilution (10-point-standard curve), and 20 μL of samples were added in duplicates to the appropriate wells. Immediately after, 100 μL of 50 ng/ml HRP-mAb (NBH250-#84) dissolved in incubation buffer (25 mM TBS-BTB, 2 g NaCl/L, pH 8, 1% Liqll) was added and the plate was incubated for 20 hours at 4° C. with shaking at 300 rpm. Following a washing step, 100 μL of CLIA substrate (ROCHE) was added, and the plate was incubated in the dark for 3 min. The RLU was measured on a SpectraMax i3x monochromator (Molecular Devices) using the luminescence plate reader. The calibration curve was plotted using a four-parametric logistic regression model with SoftMax Pro7.

### Technical Evaluation

The technical validation procedure was performed to ensure specificity, precision, linearity, accuracy, stability, and no potential interference of the assay. Requirements for the technical validation parameters were set out beforehand and were based on recognized standardized requirements for quantitative biomarker assays such as ELISAs (14). The specificity of the mAb for the standard peptide was evaluated by testing the ability of similar peptides to inhibit the signal of the binding reaction between the coater peptide and the antibody. This was tested to exclude the cross-reactivity with an elongated peptide NDAPGQYGAYF (SEQ ID NO: 2), truncated peptide_APGQYGAYF (SEQ ID NO: 3), de-selection peptide GAPGQYGEKG (SEQ ID NO: 9) (from collagen type VI αV), as well as a non-sense standard peptide (PGMRGMPGSP-GGPGSDGKPGPPGSQGESGR (SEQ ID NO: 10)) and non-sense coater (PGMRGMPGSP-LYS-biotin (SEQ ID NO: 11)), from collagen type Ill. A standard two-fold serial dilution of the 30AA LG3 standard peptide was evaluated against a two-fold serial dilution of the other similar peptides.

The precision of the assay is defined as the variation of the measured analyte in repeated measurements in samples from the same healthy donor. The precision of the assay was evaluated through intra- and inter-assay variations measured in samples from all intended matrices in ten individual runs. The intra-assay variation was calculated as the mean coefficient of variation (CV %) within plates and the inter-assay variation was calculated as the mean CV % between the plates. The acceptance limit set to 10% and 15% respectively.

Minimum required dilution (MRD) and linearity was determined by a two-fold dilution series of samples from all matrices. The recovery percentage (RE %) was then calculated relative to undiluted samples to establish linearity. To determine the stability of the analyte, human samples from all matrices was exposed to repeated freeze/thaw cycles with the first cycle as reference. To further test the stability of the analyte, samples was stored for up to 48 h at 4° C. and 20° C. and compared to a reference sample stored at −20° C. Interference was evaluated by spiking with different concentrations of haemoglobin, lipid, and biotin and comparing them to a non-spiked reference. Concentrations of haemoglobin were 2.5 and 5.0 mg/mL, lipid was 1.5 and 5.0 mg/mL, and biotin was 5, 10, 20, 40, 60, 80, and 100 ng/mL. The acceptable recovery percentage for accuracy, linearity, interference, and analyte stability tests was ±20%.

To ensure the generation of the fragment by BMP-1, a cleavage experiment was conducted with recombinant human endorepellin/perlecan (R&Dsystems, catalog Number: 2364-ER-050) and Recombinant Human BMP-1/PCP (R&Dsystems, catalog Number: 1927-ZN). Endorepellin and BMP-1 was mixed in a 1:10 (enzyme:protein) ratio with a 25 mM Hepes and 400 mM ammonium sulfate cleavage buffer and incubated for 24 hours at 37° C., 300 rpm. The setup included cleavage vials (BMP-1+endorepellin) and controls (BMP-1+buffer, endorepellin+buffer, and buffer alone). The cleavage was then stopped by adding 1 uM EDTA and all samples were then measured by the LG3 ELISA to evaluate the recognition and concentration of the cleaved fragment vs controls.

### Patient Samples

The LG3 assay was evaluated in serum and urine samples in two studies including patients with Lupus Nephritis (LN) and IgA nephropathy (IgAN). The studies were performed in collaboration with Department of Nephrology, 1st Faculty of Medicine and General University Hospital, Charles University, Prague, Czech Republic. Moreover, LG3 was measured in serum samples from the DECADE panel, a healthy reference panel including a group of samples from different age decades, and in urine samples of healthy human donors of different age and gender. Sex and mean age of the patients are shown in Table 1. Kidney biopsies from the LN patients were collected for histological assessment and evaluated for classification of glomerulonephritis (class 1-VI) as well as divided into diffuse segmental or global type IV LN, which indicate the proportion of glomeruli with fibrinoid necrosis and cellular crescent. Type V may occur in combination with type III and IV in which case both will be diagnosed (15). Class IV LN is the most commonly found as well as being the class with the worst prognosis. In addition, the global type IV LN is associated with a higher risk of end-stage renal disease than the segmental (16,17).

TABLE 1
Demographics for samples measured
with Nordic Bioscience LG3 ELISA

| Variables | Healthy | LN | IgAN |
|---|---|---|---|
| N, serum | 65 | 48 | 85 |
| Sex, (% women) | 60 | 69 | 36 |
| Age, mean (range) | 38 (20-59) | 40 (18-68) | 43 (32-54) |
| N, Urine | 48 | 52 | — |
| Sex, (% women) | 44 | 67 | — |
| Age, mean (range) | 34 (6-61) | 41 (18-68) | — |

(Healthy = healthy controls, LN = Lupus Nephritis, IgAN = IgA nephropathy)

To be able to compare the relevance of the novel LG3 assay, the same LN samples were measured with the uPERLA ELISA as well as with a commercial endorepellin ELISA (RayBiotech Human Endorepellin ELISA kit). The sex and mean age of the healthy controls used in these further measurements are shown in Table 2.

TABLE 2
Demographics for samples measured
with RayBiotech Endorepellin ELISA

| Variables | Healthy | LN |
|---|---|---|
| N, serum | 32 | 48 |
| Sex, (% women) | 64 | 69 |
| Age, mean (range) | 34 (22-48) | 40 (18-68) |
| N, Urine | 18 | 52 |
| Sex, (% women) | 55 | 67 |
| Age, mean (range) | 30 (22-42) | 41 (18-68) |

(Healthy = healthy controls, LN = Lupus Nephritis)

For sample measurements below the lower limit of quantification (LLOQ) of the specific assay, the LLOQ value is reported. All measured values from the urine samples were corrected for serum creatinine (sCr) levels measured with the QuantiChrom™ Creatinine Assay Kit (BioAssay Systems).

### Statistics

Statistical analysis and graphs were made using GraphPad Prism (version 9.2.0). Comparison of LG3 levels in three or more groups was done using a non-parametric one-way ANOVA analysis (Kruskal-Wallis test), followed by Dunn's multiple comparison test. For the comparison of the two groups, diseased and healthy donors, the non-parametric t-test (Mann Whitney test) was used. Asterisks indicate the following significance levels: * p<0.05; ** p<0.01; *** p<0.001; **** p<0.0001 and a p-value<0.05 was considered statistically significant (ns=non-significant). Spearman's correlation test was used for generating correlation coefficients (r).

### Example 2

### Results

### Technical Evaluations

In conclusion the developed competitive ELISA (i.e. the LG3 assay described above), targeting the LG3 domain of perlecan cleaved by BMP-1, met the technical requirements. The purpose of the technical validation was to ensure no unspecific binding, specificity, precision, linearity, accuracy, stability, and no interference. Requirements for the technical validation parameters were set out beforehand and were based on recognized standardized requirements for quantitative biomarker assays such as ELISAs (14).

The specificity of the mAb for the LG3 standard peptide was demonstrated as no reactivity was detected with other peptides than the standard peptide, as seen in FIG. 2, indicating that the mAb is specific towards the target of interest.

The assay was precise demonstrated by a 4% intra-assay variation and 9% inter-assay variation. The assay was stable demonstrated by consistent measurements on samples that had undergone extended incubation at different temperatures and had undergone multiple freeze-thaw cycles. Moreover, the kit was stable, demonstrated by consistent measurements on samples after the kit reagents had undergone extended incubation at room temperature. The stability of the LG3 assay is important for the future of the assay. Linearity was attained demonstrated by acceptable dilution linearity (recovery) in human serum, EDTA plasma, heparin plasma, and urine. The possibility of being able to assess LG3 in both human serum, plasma, and urine is of great advantage since there are marked differences in the protein profiles of serum and plasma. Moreover, no interference was observed for LG3 with low or high concentrations of biotin, intralipids, and hemoglobin in serum. Potential interference of salt in urine was also tested, and no interference was found. See Table 2 for the full data.

TABLE 3
Technical summary of the LG3 ELISA.

| TECHNICAL SUMMARY | LG3 PERFORMANCE |
|---|---|
| Species possibilities | Human, Mouse, Bovine |
| Standard A | 12.5 (ng/ml) |
| Control 1 | 0.9 (0.54-1.26) (ng/ml) |
| Control 2 | 3.3 (1.98-4.62) (ng/ml) |
| Intended matrix | Human serum (1:1 → 1:2) |
| (MRD and accepted dilution) | Human plasma heparin (1:1 → 1:2) |
| | Human plasma EDTA (1:4 → 1:8) |
| | Human urine (1:2 → 1:16) |
| Intra-assay CV % | Mean: 4% |
| Inter-assay CV % | Mean: 9% |
| Measurement range LLOQ - | Serum: 0.35-12.5 ng/ml (uncorrected) |
| ULOQ | Plasma Heparin: 0.1-12.5 ng/ml |
| | (uncorrected) |
| | Plasma EDTA: 0.16-12.5 ng/ml |
| | (uncorrected) |
| | Urine: 0.12-12.5 ng/ml (uncorrected) |
| Lower limit of blank (LLOB) | 0.15 ng/ml |
| Mean Slope | 1.04 |
| Mean IC25 | 0.4 ng/ml |
| Mean IC50 | 1.16 ng/ml |
| Mean IC75 | 3.39 ng/ml |
| Curve fit model | 4-point logistic curve fit |
| Accepted maximum freeze- | 5 FT (serum and plasma EDTA) |
| thaw (sample) | 2 FT cycles (plasma heparin and urine) |
| Accepted maximum stress | 4 H at 4° C. (serum and plasma EDTA) |
| (sample) | 48 H at 4° C. (plasma heparin and urine) |
| | 24 H at 20° C. (serum and plasma EDTA) |
| | 48 H at 20° C. (plasma heparin and urine) |
| Potential interference | Lipid and hemoglobin accepted |
| | Biotin accepted up to 20 ng/ml |

CV % within ± 20% was regarded as acceptable
h: hours,
C: Celsius,
FT: freeze-thaw

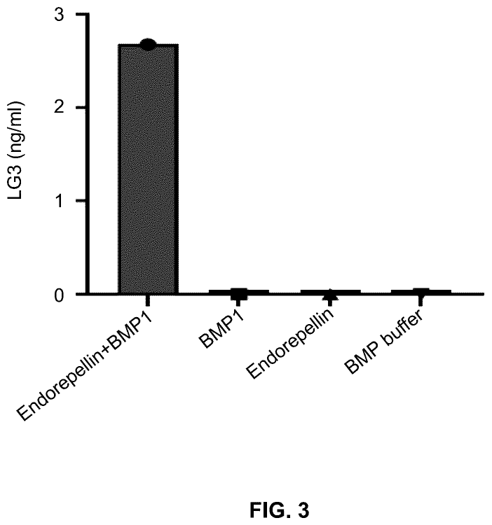

The specificity of the cleavage site and recognition by the mAb developed was confirmed by the cleavage experiment with recombinant endorepellin and BMP-1 as shown in FIG. 3.

Increased Levels of LG3 in Patients with Lupus Nephritis and IqA Nephropathy

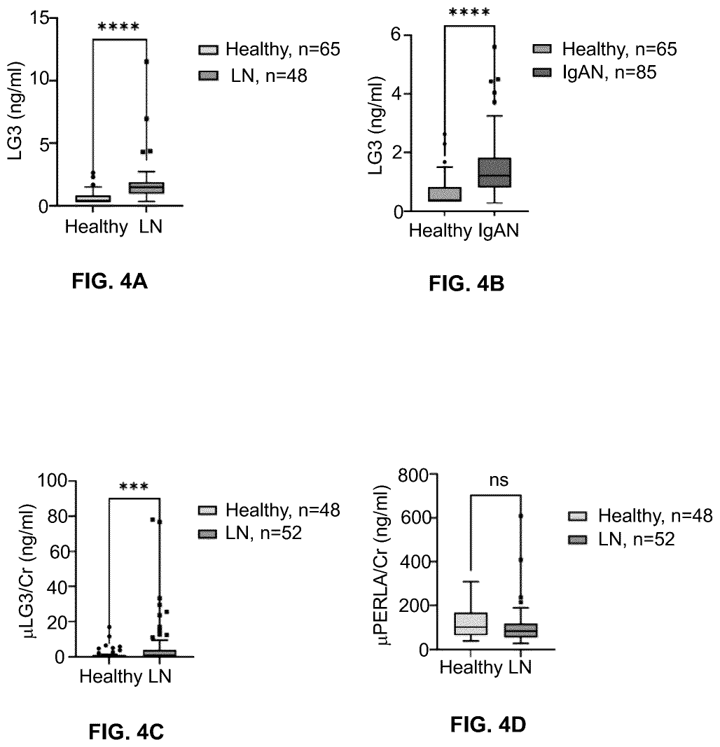

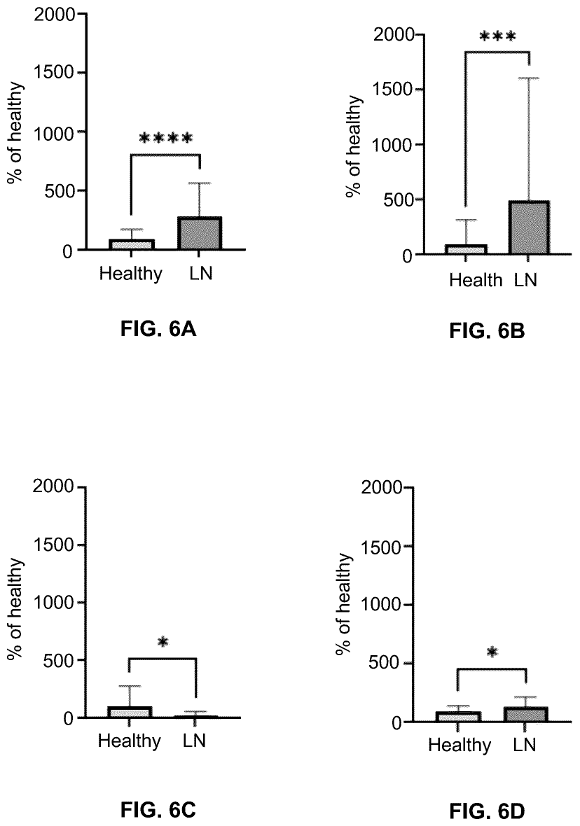

The levels of LG3 in serum as measured by the LG3 assay were significantly elevated in patients with LN (P<0.0001) and IgAN (P<0.0001) compared to healthy controls, FIGS. 4A-4B. Levels of LG3 in urine as measured by the LG3 assay (corrected for urinary creatinine) were significantly elevated in patients with LN (P<0.01) compared to healthy controls, FIG. 4C. The uPERLA assay could not significantly differentiate between LN patients and healthy controls, FIG. 4D. However, a tendency of decreased levels of uPERLA in LN patients compared to healthy may be observed. The RayBiotech Endorepellin ELISA could differentiate between LN patients and healthy controls, FIGS. 6C-6D, but to a much lesser extent than the LG3 assay, FIGS. 6A-6B. In the serum samples the RayBiotech Endorepellin ELISA detected increased levels of endorepellin in healthy samples as compared to LN patients (which is opposite to the levels of LG3 detected using the LG3 assay), and in the urine samples the RayBiotech Endorepellin ELISA detected increased levels in LN patients as compared to healthy controls (similar to the increased levels of LG3 detected by the LG3 assay, but to a much lesser extent).

These results suggest that liberation of LG3 is associated with kidney diseases and demonstrates the diagnostic potential of the LG3 assay, while substantiating the novelty and relevance of the LG3 assay independent of or in combination with uPERLA.

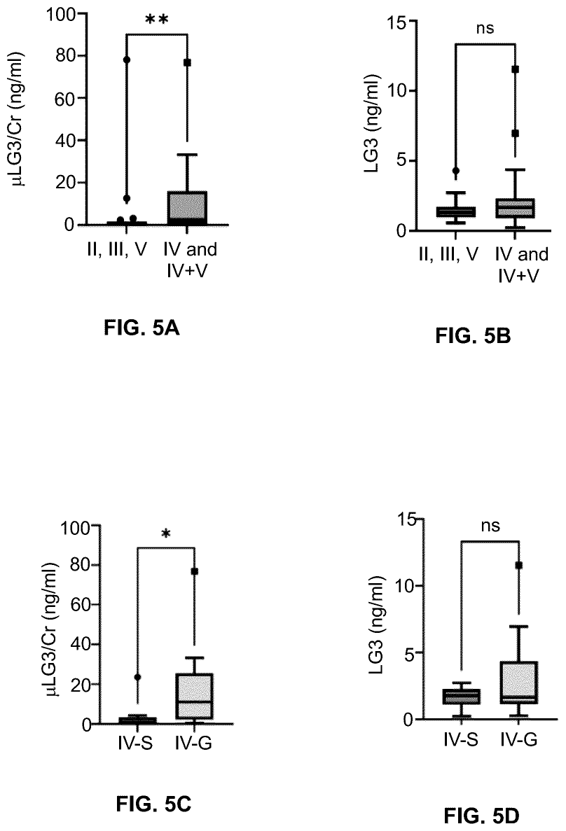

Urine LG3 levels were also significantly increased in class IV/IV+V LN patients compared to class I,II, and II grouped, FIGS. 5A-5B. When further dividing the type IV/IV+V patients into segmental or global LN, urinary LG3 levels were significantly increased in the global subtype, although serum LG3 levels were not significantly different in the same grouping or subdivision of the LN classes, FIGS. 5C-5D.

### CONCLUSION

The LG3 assay was demonstrated to be technically robust, specific, and accurate. The assay was validated in healthy human donor serum, urine, plasma heparin and EDTA samples. Moreover, the potential of LG3 as a diagnostic biomarker was demonstrated as significantly increased serum and urine levels of LG3 were detected in lupus nephritis (LN) and increased serum levels were detected in patients with IgA nephropathy (IgAN), compared to healthy controls was. The uPERLA assay showed no significant difference between LN patients and healthy controls but if any the tendency was decreased levels in diseased samples. This substantiated the opposing roles of the fragments in vivo and demonstrates a potential value in measuring both markers. In addition, LG3 was able to identify patients with a more severe type of LN, as increased LG3 levels were observed in urine samples from class IV+IV/V-global LN patients. This observation was not significant in the serum samples which could be an indication that the fragment originates from the kidney and not circulation from other organs. Together, these findings demonstrate a diagnostic role for the LG3 assay in evaluating IgA nephropathy and LN independent of intact perlecan or endorepellin.

### REFERENCES

1. Lord et al. Matrix Biol., 68-69:150-166, 2018.
2. Mongiat et al. J. Biol. Chem., 278(6):4238-4249, February 2003.
3. Bix et al. J. Cell Biol., 166(1):97-109, 2004.
4. Gonzalez et al. J. Biol. Chem., 280(8):7080-7087, 2005.
5. Oda et al. Clin. Chim. Acta, 255(2):119-132, 1996.
6. Soulez et al. Circ. Res., 110(1):94-104, 2012.
7. Surin et al. Proteomics, 13(1):142-152, January 2013.
8. Normand et al. Lupus Sci. Med., 8(Suppl 2):A12.1-A12, November 2021.
9. Chang et al. Proteomics—Clin. Appl., 2(1):23-32, 2008.
10. Karsdal et al. Clinical Biochemistry, 43(10-11):793-804, 2010.
11. Genovese et al. Fibrogenes. Tissue Repair, 7(1):1-14, 2014.
12. G. Köhler and C. Milstein, Nature, 256(5517):495-497, 1975.
13. K. Rajewsky, Nature, 575(7781):47-49, 2019.
14. Shah et al. Pharm. Res., 17(12):1551-1557, 2000.
15. Weening et al. J. Am. Soc. Nephrol., 15(2):241-250, February 2004.
16. Mahajan et al. Lupus, 29(9):1011, August 2020.
17. Roth et al. J. Am. Coll. Cardiol., 76(25):2982-3021, 2020.

What is claimed is:

1. A method of immunoassay, said method comprising;
i) contacting a patient sample with a monoclonal antibody that specifically binds to an N-terminus amino acid sequence DAPGQYGAYF (SEQ ID NO: 2); and
ii) detecting and determining an amount of binding between said monoclonal antibody and peptides in the sample.

2. The method as claimed in claim 1, wherein the method is a method of immunoassay for detecting and/or monitoring a fibrotic kidney disease or a particular level of severity thereof in a patient, the method further comprising;
iii) correlating said amount of binding with values associated with normal healthy subjects and/or values associated with a known disease severity and/or values obtained from said patient at a previous time point and/or with a predetermined cut-off value.

3. The method as claimed in claim 2, wherein the fibrotic kidney disease is Lupus Nephritis or IgA nephropathy.

4. The method as claimed in claim 3, wherein the fibrotic kidney disease is Lupus Nephritis.

5. The method as claimed in claim 4, wherein the method is a method for detecting a particular level of severity of Lupus Nephritis in a patient.

6. The method as claimed in claim 1, wherein the patient sample is selected from blood, plasma, serum or urine.

7. The method as claimed in claim 6, wherein the patient sample is a urine sample.

8. The method as claimed in claim 1, wherein the monoclonal antibody does not specifically bind to a peptide having an N-terminus amino acid sequence NDAPGQYGAYF (SEQ ID NO: 3).

9. The method as claimed in claim 1, wherein the monoclonal antibody does not specifically bind to a peptide having an N-terminus amino acid sequence APGQYGAYF (SEQ ID NO: 4).

10. The method as claimed in claim 1, wherein the monoclonal antibody is raised against a synthetic peptide having the N-terminus amino acid sequence DAPGQYGAYF (SEQ ID NO: 2).

11. The method as claimed in claim 1, wherein the immunoassay is a competition assay or a sandwich assay.

12. The method as claimed in claim 1, wherein the immunoassay is a radio-immunoassay or an enzyme-linked immunosorbent assay.

13. A monoclonal antibody that specifically binds to an N-terminus amino acid sequence DAPGQYGAYF (SEQ ID NO: 2).

14. The monoclonal antibody as claimed in claim 13, wherein the monoclonal antibody does not specifically bind to a peptide having an N-terminus amino acid sequence NDAPGQYGAYF (SEQ ID NO: 3).

15. The monoclonal antibody as claimed in claim 13, wherein the monoclonal antibody does not specifically bind to a peptide having an N-terminus amino acid sequence APGQYGAYF (SEQ ID NO: 4).

16. The monoclonal antibody as claimed in claim 13, wherein the monoclonal antibody is raised against a synthetic peptide having the N-terminus amino acid sequence DAPGQYGAYF (SEQ ID NO: 2).

17. An immunoassay kit comprising a monoclonal antibody as claimed in claim 13, and at least one of:
a streptavidin coated well plate;
a biotinylated peptide DAPGQYGAYF-L-Biotin (SEQ ID NO: 5), wherein L is an optional linker;
a secondary antibody for use in a sandwich immunoassay;
a calibrator protein comprising the N-terminus amino acid sequence DAPGQYGAYF (SEQ ID NO: 2);
an antibody biotinylation kit;
an antibody HRP labelling kit; or
an antibody radiolabelling kit.


SEQUENCE LISTING (11 sequences)

SEQ ID NO: 1 - protein, 10 aa, synthetic construct
SIEYSPQLED

SEQ ID NO: 2 - protein, 10 aa, synthetic construct
DAPGQYGAYF

SEQ ID NO: 3 - protein, 11 aa, synthetic construct
NDAPGQYGAYF

SEQ ID NO: 4 - protein, 9 aa, synthetic construct
APGQYGAYF

SEQ ID NO: 5 - protein, 10 aa, synthetic construct
  MOD_RES 10: note = linked to biotin optionally via a linker
DAPGQYGAYF

SEQ ID NO: 6 - protein, 13 aa, synthetic construct
  MOD_RES 13: note = linked to Keyhole Limpet Hemocyanin
DAPGQYGAYFGGC

SEQ ID NO: 7 - protein, 30 aa, synthetic construct
DAPGQYGAYFHDDGFLAFPGHVFSRSLPEV

SEQ ID NO: 8 - protein, 11 aa, synthetic construct
  MOD_RES 11: note = biotinylated
DAPGQYGAYFK

SEQ ID NO: 9 - protein, 10 aa, synthetic construct
GAPGQYGEKG

SEQ ID NO: 10 - protein, 30 aa, synthetic construct
PGMRGMPGSPGGPGSDGKPGPPGSQGESGR

SEQ ID NO: 11 - protein, 13 aa, synthetic construct
  MOD_RES 13: note = biotinylated
PGMRGMPGSPLYS